(19) United States
(12) Patent Application Publication
(10) Pub. No.: US 2010/0168547 A1
(43) Pub. Date: Jul. 1, 2010

(54) UNIVERSAL DIN LEADWIRE SYSTEM FOR USE WITH EKG AND ECG PATIENT MONITORING AND EVENT RECORDING INSTRUMENTS

(76) Inventor: David Kendricks, Coral Springs, FL (US)

Correspondence Address: MALIN HALEY DIMAGGIO BOWEN & LHOTA, P.A., 1936 S ANDREWS AVENUE, FORT LAUDERDALE, FL, 33316, US

(21) Appl. No.: 12/344,764

(22) Filed: Dec. 29, 2008

(51) Int. Cl. A61B 5/0408

(52) U.S. Cl. 600/372; 600/382

(57) ABSTRACT

A universal DIN leadwire system including adapters and a leadwire combiner that is configurable for use with EKG and ECG monitoring and event recording equipment having specially configured yoke assemblies is disclosed. Disposable standardized DIN leadwire subassemblies are provided such that leadwires are always immediately available for use with virtually any EKG and ECG monitoring and event recording equipment found in the health care field. Individual DIN leadwires are combined and electrically connected to a combiner. The combiner includes a connectable end having projecting electrical connectors fabricated in conformance with acceptable electrical standards for such equipment. Various adapters provide an interface between the universal combiner and a specific make and/or model of EKG or ECG monitoring and/or event recording instrument. Each adapter has an end configured for mating electrical connection with the universal combiner leadwire subassembly. Each adapter further includes an opposing end having projecting electrical connectors that have been configured by size, shape, number, key configuration, location etc. for mating engagement with the yoke assembly of a particular make or model EKG or ECG monitoring and/or event recording instrument.

### CROSS REFERENCE TO RELATED APPLICATIONS

N/A

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all rights whatsoever.

### BACKGROUND OF THE INVENTION

1. Field of the Invention

The present invention relates generally to electrode leadwire systems used in EKG and ECG monitoring, telemetry, and event recording systems and instruments, and more particularly to a universal DIN leadwire system including adapters and an universal leadwire combiner system to allow for adaptable electrical connection and use of electrode leads with various EKG and ECG monitoring, telemetry, and event recording equipment.

2. Description of Related Art

An electrocardiogram (ECG or EKG, abbreviated from the German Elektrokardiogramm) is a graphic produced by an electrocardiograph, which records the electrical voltage in the heart in the form of a continuous strip graph. It is the prime tool in cardiac electrophysiology, and has a prime function in screening and diagnosis of cardiovascular diseases.

The ECG has a wide variety of uses including: (1) determining whether the heart is performing normally or suffering from abnormalities; (2) indicating acute or previous damage to heart muscle (heart attack) or ischaemia of heart muscle (angina); (3) detecting potassium, calcium, magnesium and other electrolyte disturbances; (4) allowing the detection of conduction abnormalities (heart blocks and bundle branch blocks); (5) as a screening tool for ischaemic heart disease during an exercise tolerance test; (6) providing information on the physical condition of the heart; and (7) providing a tool to diagnose or suggest non-cardiac disease.

An ECG is constructed by measuring electrical potential between various points of the body using a galvanometer. In the United States, leads I, II and III are measured over the limbs: I is from the right to the left arm, II is from the right arm to the left leg and III is from the left arm to the left leg. From this, the imaginary point V is constructed, which is located centrally in the chest above the heart. The other nine leads are derived from potential between this point and the three limb leads (aVR, aVL and aVF) and the six precordial leads (V₁₋₆).

Therefore, there may be as many as twelve leads in total. Each lead, by its nature, records information from a particular part of the heart. The inferior leads (leads II, III and aVF) look at electrical activity from the vantage point of the inferior region (wall) of the heart. This is the apex of the left ventricle. The lateral leads (I, aVL, V₅ and V₆) look at the electrical activity from the vantage point of the lateral wall of the heart, which is the lateral wall of the left ventricle. The anterior leads, V₁ through V₆, and represent the anterior wall of the heart, or the frontal wall of the left ventricle. An aVR is rarely used for diagnostic information, but indicates if the ECG leads were placed correctly on the patient.

The EKG and ECG monitoring and event recording instruments in common use rely on DIN or other connectors to facilitate electrical connection between the electrodes and the monitoring and/or recording instrument. A DIN connector is a connector that was originally standardized by the Deutsches Institut für Normung (DIN), the German national standards organization. Most EKG and ECG instruments include a yoke assembly including a trunk cable that is connected to the instrument. The yoke assembly provides, at its other end, a DIN electrical connector for connection to a DIN leadwire set. DIN leadwires are typically non-shielded however, shielded (i.e. shielded from electromagnetic interference) leadwires or connections are also used in the field. A typical DIN connection includes a first connector component having a recessed pin and a second connector component having a socket for mating electrical engagement with the pin.

A significant problem involves the absence of standards governing the electro-mechanical connection aspects in this field. As a result, the various manufacturers of EKG and ECG monitoring and event recording instruments specifically design the yoke assembly to include a connector with a specific shape and/or key configuration in an effort to ensure that only the manufacturers original leadwire equipment can be used with its monitoring and event recording instruments. Accordingly, manufacturers produce yoke and leadwire electrical connections in various shapes and configurations thereby limiting interconnection of components, particularly components produced by other manufacturing sources. Manufacturers typically key electrical connectors so as to further polarize the connection. In addition, the number of leadwire electrical connections varies between various makes and models of equipment. Furthermore, the lack of standardization has resulted in the proliferation of differing color code schemes used to identify particular electrodes. The use of such specialized connection configurations renders it difficult for health care workers to quickly replace broken, damaged, or lost DIN leadwire sets. In addition, the DIN leadwire sets typically provided by the original equipment manufacturer are often non-disposable, and thus must be thoroughly cleaned and disinfected between uses thereby increasing costs.

As a result of these limitations, there exists a need for a universal DIN leadwire system including adapters and a leadwire combiner to allow for use of DIN leadwires with EKG and ECG monitoring and event recording equipment of various manufactured configurations. There further exists a need for such a system that provides disposable DIN leadwires.

### BRIEF SUMMARY OF THE INVENTION

The present invention overcomes the limitations and disadvantages present in the art by providing a universal DIN leadwire system including leadwire combiner and adapter technology configurable for use with EKG and ECG monitoring, telemetry, and other patient event recording equipment having specially configured yoke assemblies. The present invention further provides such a universal system having disposable DIN leadwire subassemblies such that disposable leadwires are always immediately available for use with virtually any EKG and ECG monitoring and event recording equipment found in the health care field.

In accordance with a first aspect of the present invention, a universal leadwire combiner is provided for receiving and securing individual leadwires. Each leadwire typically includes a first or input end terminating in an attachment for an electrode and a second or output end terminating in an electrical connector, such as DIN connector, or any other suitable electrical connector, for connection to the yoke cable assembly of an EKG, ECG, or telemetry device used in patient monitoring. The leadwires may be shielded or non-shielded, radiolucent, or any other suitable leadwire structure. A combiner is provided and adapted to receive a predetermined number of leadwires in a combined configuration with the leadwire first end projecting from one end of the combiner and the lead wire second end connector projecting from the other connectable outlet end of the combiner forming an electrical connector. The combiner outlet end may be specifically configured and/or keyed to allow for the combiner to be received within a specially configured electrical connector on the original yoke cable provided with an EKG or ECG instrument originating from a specific manufacturer. The combiner connectable end thus includes projecting male electrical connectors fabricated in conformance with acceptable electrical standards for such equipment to allow for the fabrication of replacement leadwire sets for use with existing hospital equipment. In addition, the combiner and leadwires may comprise a sub-system that is disposable. Accordingly, the combiner can be adapted to receive any number of leadwires (e.g. 3, 5, 6, 8, 10, 12, 14 etc.). The present invention thus provides a leadwire combiner that is adaptable for use with any electrical connector configuration, and is particularly suitable for providing disposable shielded on non-shielded DIN leadwire sets.

In accordance with a second aspect of the present invention, a single/universal combiner structure is provided with various adapters her provided to allow for interface between the universal combiner and the yoke assembly of a specific make and/or model of EKG or ECG monitoring and/or event recording instrument. Each adapter has a female connection configured for mating electrical connection with the universal combiner and leadwire subassembly. Each adapter further includes projecting male electrical connectors that have been configured by size, shape, number, key configuration, location etc. for mating engagement with the yoke assembly of a particular make or model EKG or ECG monitoring and/or event recording instrument. Adapters in accordance with the present invention allow for the connection and use of leadwires in the universal combiner with the yoke assembly of a particular make or model EKG or ECG monitoring and/or event recording instrument.

The present invention thus provides a universal leadwire system for universal use with any EKG or ECG monitoring, telemetry, and/or event recording instruments. As a result, healthcare providers have ready access to shielded and non-shielded disposable leadwire assemblies (e.g. DIN leadwire sets) that are adaptable for use with differing equipment configurations. As noted above, the leadwires may be DIN, or other DIN-like type, and may further be shielded or non-shielded, and may further have radiolucent characteristics. The present invention simplifies inventory and logistics by ensuring a relatively low cost disposable DIN leadwire systems are on hand and available for use with any monitoring or event recording instrument employed for patient care.

Accordingly, it is an object of the present invention to provide a universal leadwire system for use with EKG and ECG monitoring, telemetry, and event recording instruments having different connection configurations.

Another object of the present invention is to provide a universal combiner adaptable for direct connection to the yoke assembly of any given make, model, or manufacturer.

Yet another object of the present invention is to provide a universal leadwire system including combiners and adapters that allow for connection and use with EKG, ECG monitoring, telemetry, and event recording instruments originating from various manufacturers.

In accordance with these and other objects, which will become apparent hereinafter, the instant invention will now be described with particular reference to the accompanying drawings.

### BRIEF DESCRIPTION OF THE SEVERAL VIEWS OF THE DRAWINGS

FIG. 1 illustrates a universal DIN (5-electrode) leadwire system, including universal combiner and multiple adapters, for use with EKG, ECG, or telemetry monitoring and event recording instruments in accordance with the present invention;

FIG. 2 is a top perspective partially exploded view of the connecting end of a 5-leadwire combiner shown with the closure open to illustrate insertion of DIN leadwires in accordance with the present invention;

FIG. 3 is a top perspective view thereof with the leadwires insertedly received in the combiner;

FIG. 4 is a top perspective view thereof with the closure in the closed position;

FIG. 5 is an end view of a combiner illustrating a particular keyed configuration;

FIG. 6 is a top perspective view of a 3 leadwire adapter in accordance with the present invention for connection to an OEM yoke assembly having six electrical connectors in a keyed 2×3 configuration;

FIG. 7 is a top perspective view of an alternate embodiment 5 leadwire adapter in accordance with the present invention having a keyed 2×5 configuration;

FIG. 8 is a top perspective view of an alternate embodiment 6 leadwire adapter;

FIG. 9 is an end view thereof.

FIG. 10 is an exploded perspective view thereof;

FIGS. 11 and 12 are top perspective views illustrating the combination of a keyed 3 leadwire combiner with an adapter in accordance with the present invention; and

FIG. 13 is a sectional perspective view thereof.

### DETAILED DESCRIPTION OF THE INVENTION

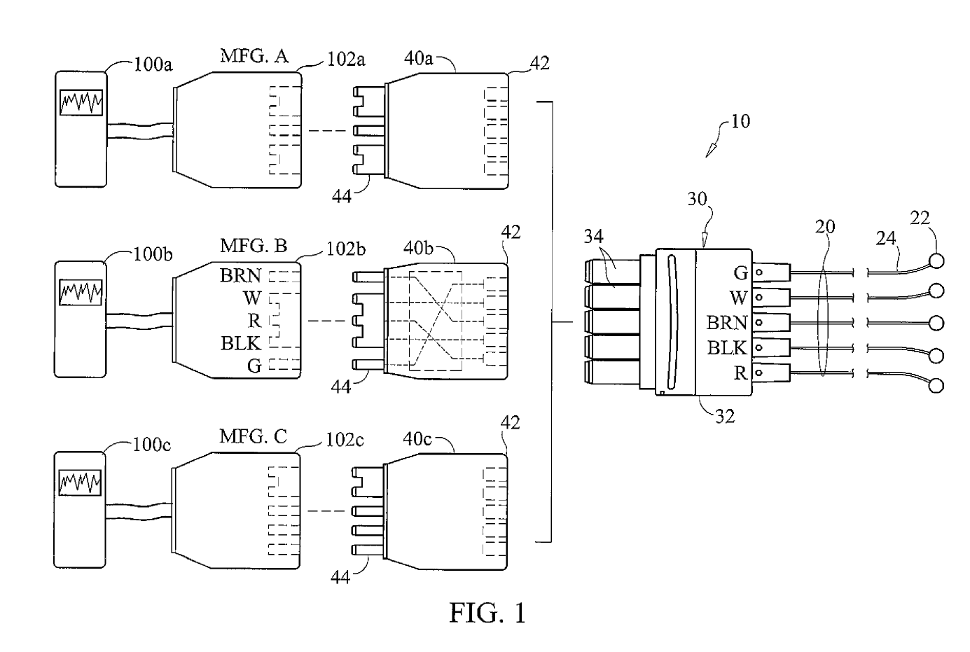

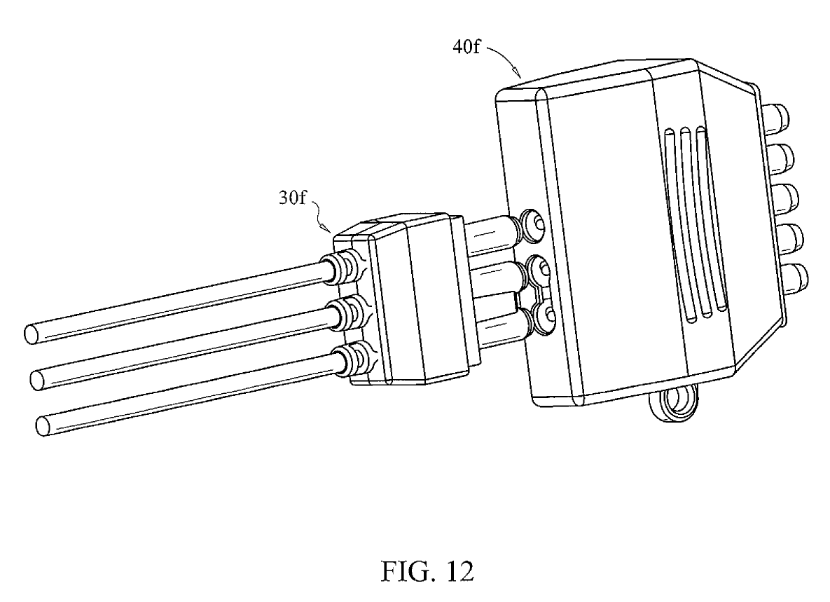

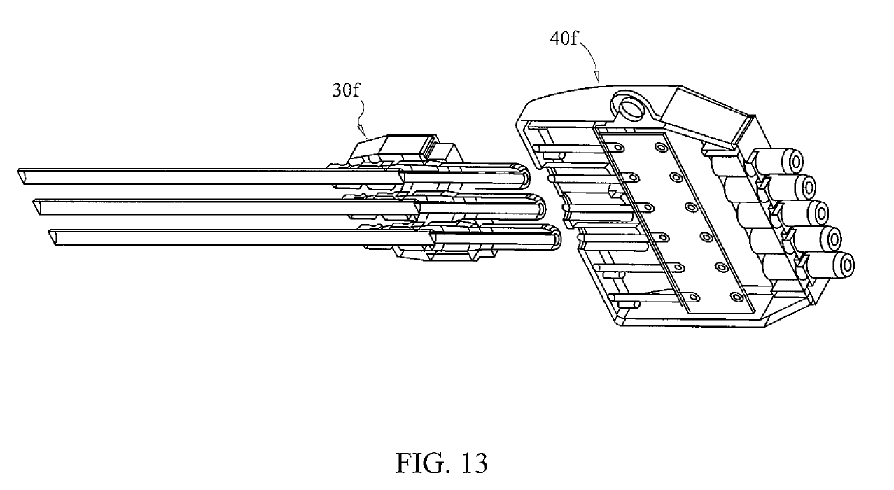

With reference to the drawings, FIGS. 1-13 illustrate and depict preferred and alternate embodiments of a universal leadwire system, generally referenced as 10, including adapters and leadwires secured by an adaptable leadwire combiner that may be configured for use with EKG, ECG, and telemetry monitoring and event recording instruments, generally referenced as 100a, 100b, and 100c, having specially configured yoke assemblies, referenced as 102a-102c.

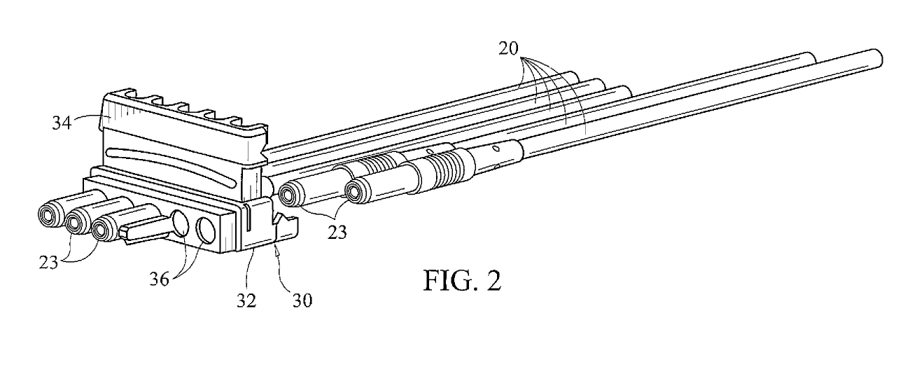

As best illustrated in FIGS. 1-4, individual DIN leadwires, generally referenced as 20, for use in EKG and ECG monitoring are disclosed with a universal DIN leadwire combiner 30. Each leadwire typically includes a first or input end 22 preferably terminating in an electrode (or electrode connector) for attachment to the patient, a second or output end 23 terminating in an electrical connector, and an insulated electrical conductor 24 in electrical communication with input end 22 and output end 23. FIGS. 2-4 illustrate leadwire output ends 23 terminating in a non-shielded DIN connector. The leadwire output ends 23, however, may comprise any other electrical connector suitable for connection to the yoke cable assembly of an EKG, ECG, or telemetry device used in patient monitoring. In addition, leadwires 20 may be shielded or non-shielded, radiolucent, or any other suitable leadwire structure. Leadwires 20 and leadwire sets may further be re-usable or disposable.

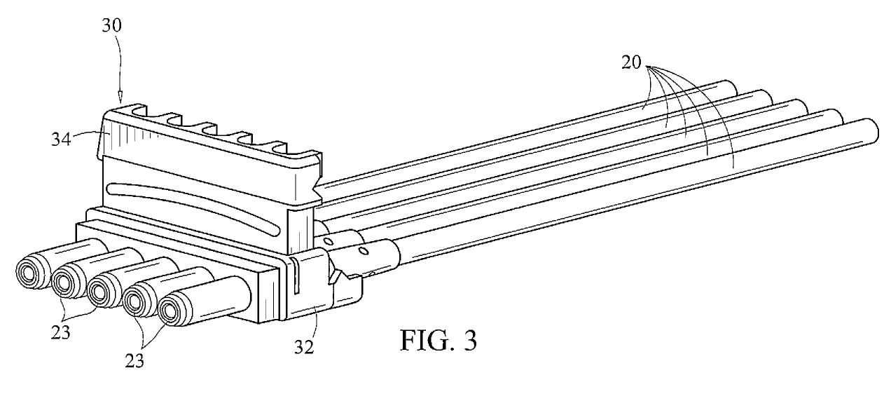

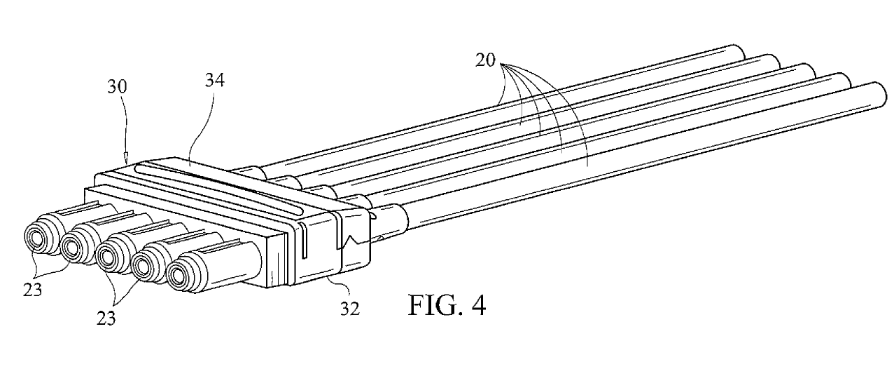

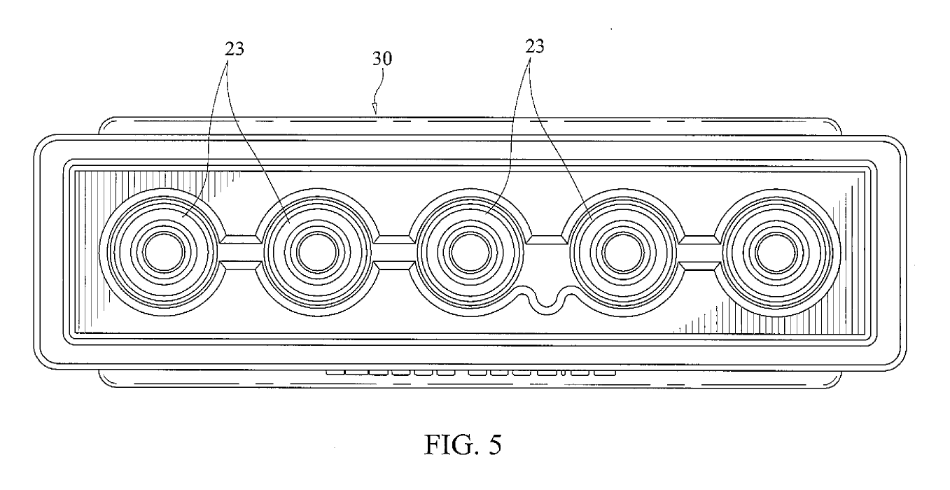

A combiner, generally referenced as 30, provides a structure securing a plurality of leadwires. Combiner 30 is preferably fabricated from plastic, however, any suitable material is considered within the scope of the present invention. Combiner 30 may be adapted to receive any number of leadwires (e.g. 3, 5, 6, 8, 10, 12, 14 etc.). Combiner 30 includes a main body 32 having an openable closure 34. Main body 32 includes an end defining a plurality of openings or apertures 36 through which lead wire output ends 23 project. FIGS. 2-4 illustrate a combiner in accordance with the present invention adapted for receiving leadwires 20 terminating in generally cylindrical DIN type connectors 23 in accordance with a preferred embodiment. Accordingly, combiner apertures 36 are generally circular and sized for receiving connectors 23. FIGS. 2 and 3 illustrate the DIN connectors 23 of leadwires 20 being received within combiner 30 such that connectors 23 project through apertures 36. FIG. 4 illustrates securing the leadwire DIN connectors 23 in combiner body 32 with the combiner closure 34 in the closed configuration. FIG. 5 depicts a specifically keyed end or a combiner 30. The present invention contemplates providing any particular keyed end configuration, including variation of shape, size, number, spacing and arrangement, etc. Accordingly, combiner 30 may be adapted to electrically mate with the yoke assemblies of various EKG and ECG machines which are often specifically structured or keyed to prevent hospital staff from incorrectly plugging in the leadwire assembly and for preventing use of the EKG systems with leadwire assemblies obtained from third parties. Accordingly, the end or combiner 30 from which connectors 23 project is keyed or otherwise structured as necessary to allow for direct connection to the specifically keyed or structured yoke assembly for any given patient monitoring system. Thus, in accordance with a first aspect of the present invention, a system of combiners and leadwires is disclosed to provide replacement leadwires for virtually any EKG or ECG monitoring and/or event measuring instrument of a particular make or model. The present invention thus provides a leadwire combiner that is adaptable for use with any electrical connector configuration, and is particularly suitable for providing disposable shielded on non-shielded DIN leadwire sets.

In accordance with another significant aspect of the present invention, a universal combiner 30 is provided and used in combination with adapters, generally referenced as 40, to facilitate connection to EKG or ECG monitoring and/or event measuring instruments originating from various manufacturers. This embodiment of the present invention simplifies and reduces hospital inventory management by allowing for the stocking of one-type of leadwire that may be used with any of the hospital's EKG and ECG monitoring and/or event measuring instruments by using an adapter 40 configured to allow for the electrical connection of the universal combiner with a particular model patient monitoring machine. This advancement is significant since ECG, EKG, and patient monitoring systems are not standardized and are thus known to have different electrical connection configurations.

RIG. 1 provides a schematic illustration of a DIN lead wire system with universal combiners 30 and variously configured adapters, referenced as 40a-40c. Each adapter 40a-40c includes projecting electrical connectors, generally referenced as 44a-44c, that have been specifically sized, shaped, keyed, positioned, or otherwise configured for direct electrical connection to the yoke assembly 102a- 102c of an EKG or ECG monitoring and/or event measuring instrument, referenced as 100a -100c, of a particular make or model. The present invention provides a universal leadwire system adaptable for connection to virtually any EKG and/or ECK monitoring and event recording instrument. More particularly, various adapters, referenced as 40a, 40b, and 40c, provide electrical interface between the universal combiner 30 and a specific make and/or model of EKG or ECG monitoring and/or event recording instrument, which instruments are referenced as 100a, 100b, and 100c, having yoke specially configured connectors referenced as 102a-102c. Each adapter 40 has a recessed female electrical connection end (e.g. having a DIN pin), referenced as 42 configured for mating electrical connection with the leadwire electrical contacts 23 of combiner 30 in electrical communication with electrodes 22 via wires 24. Each adapter further includes projecting male electrical connectors 44 (e.g. having the DIN socket) that have been configured by size, shape, number, key configuration, location etc. for mating engagement with the yoke assembly 102 of a particular make or model EKG or ECG monitoring and/or event recording instrument as illustrated in FIG. 1.

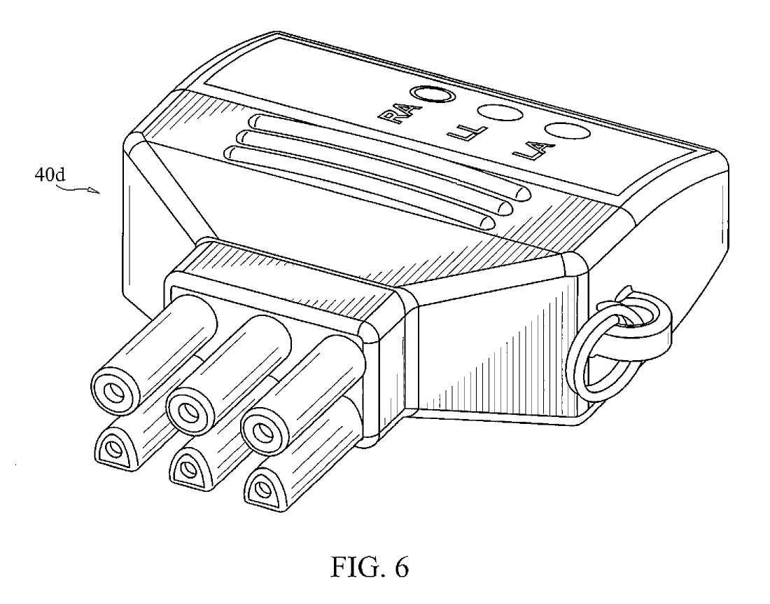

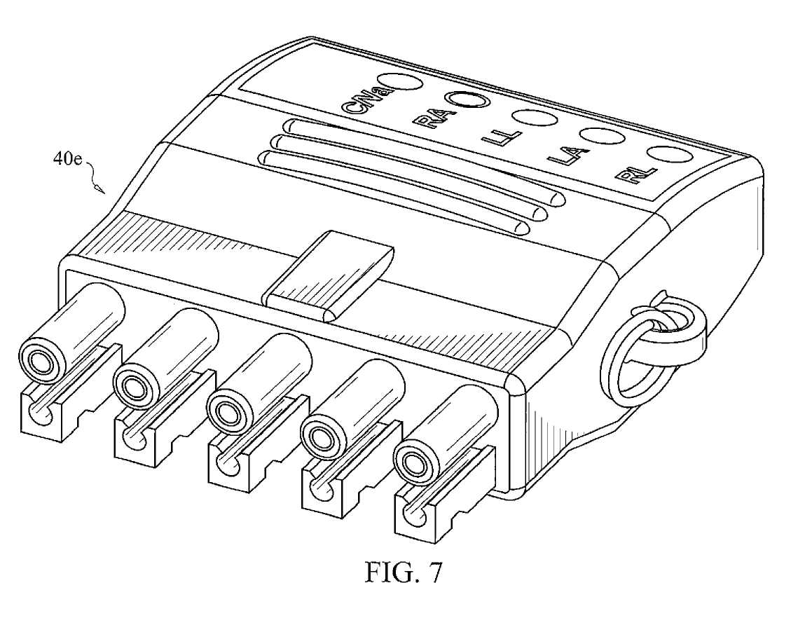

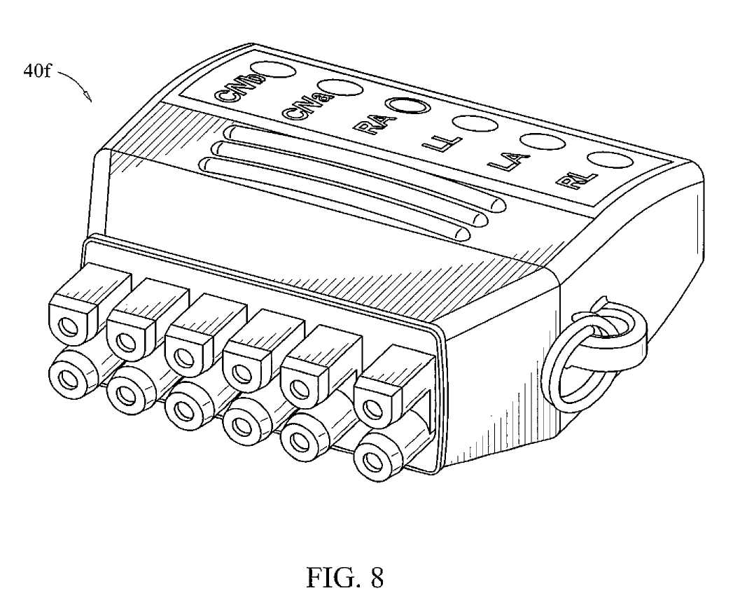

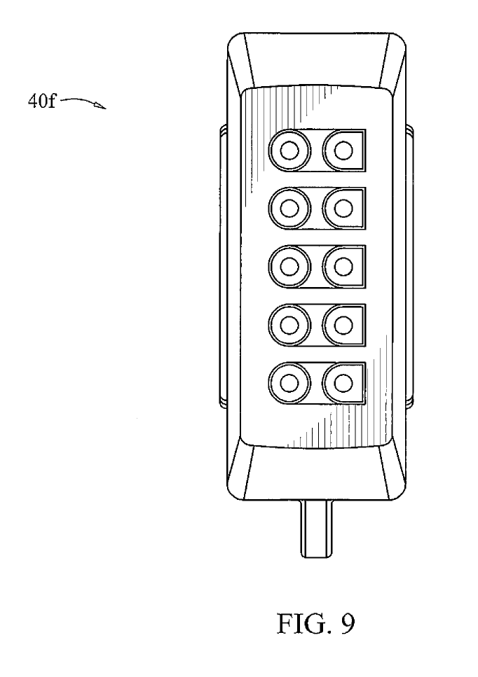

Since there exist no standard for color coding of leadwires, and in fact the various manufacturers have adopted widely varying color coding schemes, the present invention contemplates the adoption and use of universal combiner 30 having a set color coding scheme wherein each leadwire is associated with a color code as illustrated in FIG. 1. While a specific color code arrangement is disclosed, the present invention should be construed to cover any variation in leadwire number or color. Each color corresponds to the intended fixation location for an electrode, e.g. right arm, right leg, left arm, left leg, and V₁-V₆ in proximity to the heart. FIG. 6 is a top perspective view of a 3 leadwire adapter, referenced as 40d, for connection to a yoke assembly having six electrical connectors in a keyed 2 (row)×3 (column) configuration in accordance with the present invention. FIG. 7 is a top perspective view of an alternate embodiment 5 leadwire adapter, referenced as 40e, in a keyed 2×5 configuration. FIG. 8 is a top perspective view of an alternate embodiment 6 leadwire adapter 40f, and FIG. 9 is an end view thereof. FIGS. 6-9 are examples of key configurations required for mating engagement of the adapters with the yoke assembly for a particular EKG, ECG, or telemetry component for an actual make and model instrument.

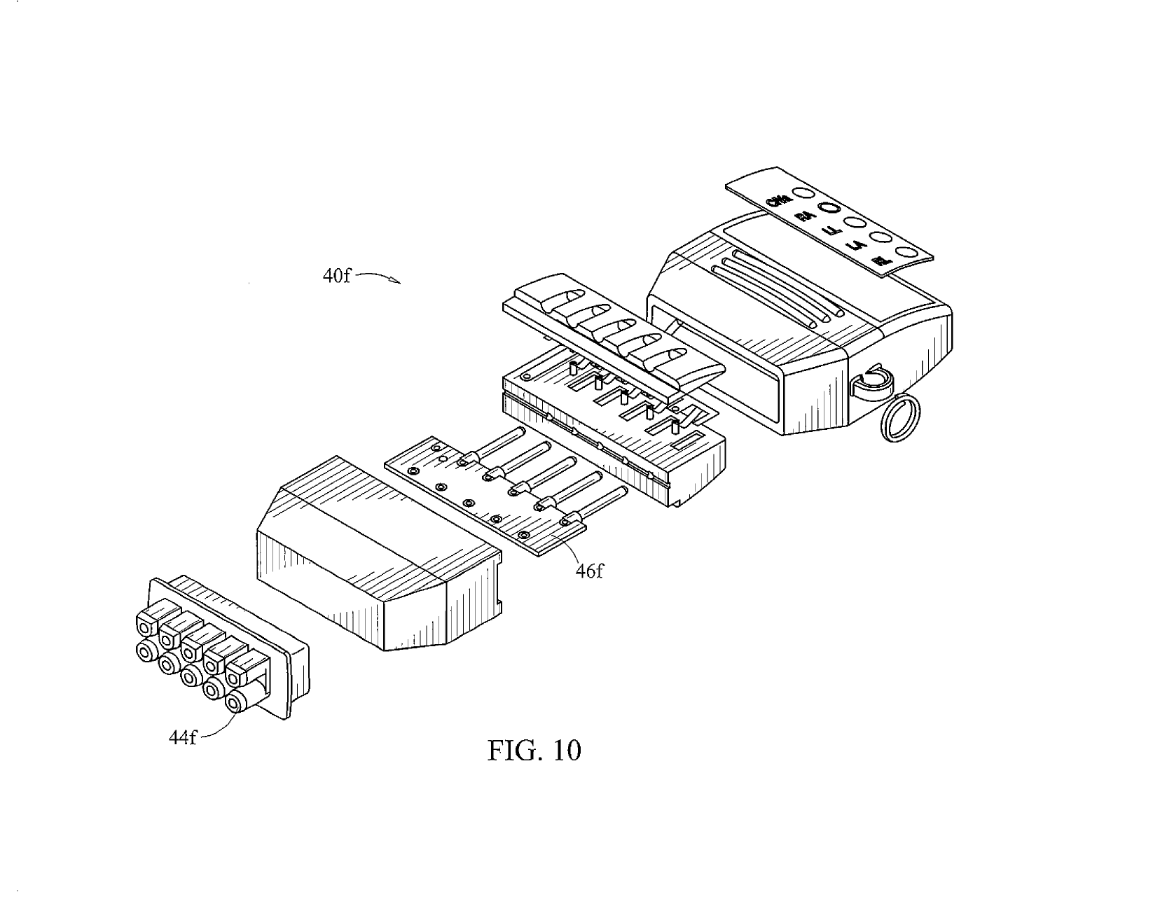

In view of the lack of standardization relating to leadwire color coding, each adapter 40 includes a printed circuit board, generally referenced as 46 having conductors thereon to redirect electrical signals between the female electrical connection end 42 and the male electrical connection end 44 as best illustrated by adapter 40b in FIG. 1, and as further shown in FIG. 10. Circuit board 46 provides a means for re-routing the leadwire configuration found on the leadwire subassembly, namely combiner 30, to the input configuration on the yoke for a particular make or model patient monitoring instrument. More particularly, as best illustrated in FIG. 1, universal combiner 30 is configured with leadwires 20 coded (from top to bottom) green (Gr), white (Wh), brown (Br), black (Bk), and red (Rd). Since the EKG or ECG instrument referenced as 100b includes a yoke assembly 102b wherein the manufacturer's inputs are coded (from top to bottom) brown (Br), white (Wh), red (Rd), black (Bk), and green (Gr), adapter 40b includes a printed circuit board 46b that functions to route the electrical connections from the combiner 30 to the suitable configuration required by yoke 102b. As a result, adapters 40 not only provide means for physically connecting the leadwires via a combiner to an EKG or ECG instrument, but further function to internally reroute the wires as necessary to function with a particular make and/or model EKG or ECG instrument.

The present invention thus provides a system for universal use with any EKG or ECG monitoring and/or event recording instrument. As a result, healthcare providers have ready access to disposable DIN leadwires that are adaptable for use with differing equipment configurations. The present invention simplifies inventory and logistics by ensuring that uncontaminated, relatively low cost disposable DIN leadwire systems are on hand and available for use with any monitoring or event recording instrument employed for patient care.

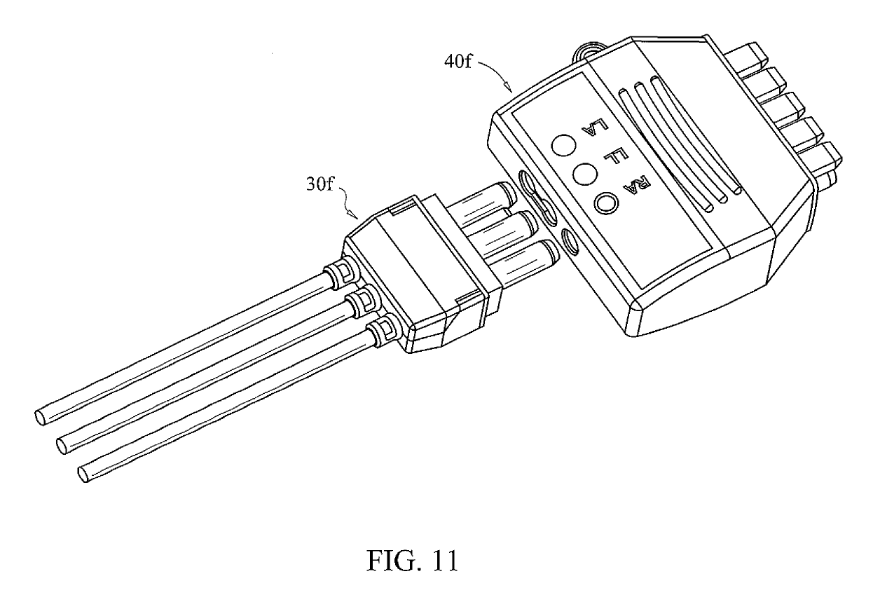

FIGS. 11-13 illustrate a further advantage of the present invention, namely, allowing the use of a 3 leadwire set with a universal combiner 30 with a patient monitoring system having a 5 wire input by using an adapter 40f in accordance with the present invention. As should now be apparent, the present invention further allows for the use of varying number of leadwires with a single patient monitoring system.

The instant invention has been shown and described herein in what is considered to be the most practical and preferred embodiment. It is recognized, however, that departures may be made therefrom within the scope of the invention and that obvious modifications will occur to a person skilled in the art.

What I claim is:

1. A universal DIN leadwire system for mating electrical connection with the trunk cable yoke of a patient monitoring apparatus the yoke terminating in a specially structured electrical connection configuration, said universal DIN leadwire system comprising:
a plurality of electrically conducting leadwires, each lead wire having a first end adapted for monitoring a patient's bio-electrical signals and a second end terminating in a DIN electrical connector;
a universal combiner having connectable end defining a plurality of apertures, said plurality of electrically conducting leadwires received in said combiner with said second end DIN electrical connectors thereof projecting from said combiner connectable end in a predetermined standard configuration; and
an adapter having a first end configured for electrical mating engagement with said combiner connectable end, and a second end configured for electrical mating engagement with the specially structured electrical connection of the yoke.

2. A universal DIN leadwire system according to claim 1, wherein said adapter includes means for electrically interfacing said DIN electrical conductors in said predetermined standard configuration with the specially structured electrical connection of the yoke.

3. A universal DIN leadwire system according to claim 2, wherein said means for electrically interfacing includes a printed circuit board having inputs corresponding to said leadwires and outputs corresponding to the yoke.

4. A universal DIN leadwire system according to claim 1, wherein said leadwires are shielded.

5. A universal DIN leadwire system according to claim 1, wherein said leadwires are non-shielded.

6. A universal DIN leadwire system for mating electrical connection with the trunk cable yokes of patient monitoring apparatus from first and second manufacturing sources, where the yoke of the apparatus from the first manufacturing source terminates in first specially structured electrical connection configuration and the yoke of the apparatus from the second manufacturing source terminates in a second specially structured electrical connection configuration, said universal DIN leadwire system comprising:
a plurality of electrically conducting leadwires, each lead wire having a first end adapted for monitoring a patient's bio-electrical signals and a second end terminating in a DIN electrical connector;
a universal combiner having connectable end defining a plurality of apertures, said plurality of electrically conducting leadwires received in said combiner with said second end DIN electrical connectors thereof projecting from said combiner connectable end in a predetermined standard configuration;
a plurality of adapters, including first and second adapters;
said first adapter having a first end configured for electrical mating engagement with said combiner connectable end, and a second end configured for electrical mating engagement with the specially structured electrical connection of the yoke from the first manufacturer; and
said second adapter having a first end configured for electrical mating engagement with said combiner connectable end, and a second end configured for electrical mating engagement with the specially structured electrical connection of the yoke from the second manufacturer.

7. A universal DIN leadwire system according to claim 1, wherein said adapter includes means for electrically interfacing said DIN electrical conductors in said predetermined standard configuration with the specially structured electrical connection of the yoke.

8. A universal DIN leadwire system according to claim 2, wherein said means for electrically interfacing includes a printed circuit board having inputs corresponding to said leadwires and outputs corresponding to the yoke.

9. A universal DIN leadwire system according to claim 1, wherein said leadwires are shielded.

10. A universal DIN leadwire system according to claim 1, wherein said leadwires are non-shielded.

11. A method of providing universal DIN leadwire system for mating electrical connection with the trunk cable yokes of patient monitoring apparatus of various makes and models, said universal DIN leadwire system comprising:
providing a DIN leadwire set for attachment to a patient for use in monitoring bio-electrical activity, said DIN leadwire set including a combiner having connectable end including DIN electrical connectors projecting therefrom in a standard configuration,
providing a plurality of adapters, each adapter having a first end configured for electrical mating engagement with said combiner connectable end, and a second end configured for electrical mating engagement with the specially structured electrical connection of the yoke for particular patient monitoring apparatus;
each adapter including means for routing electrical communication between said DIN electrical connectors and the specially structured electrical connection of the yoke for particular patient monitoring apparatus.

12. A universal DIN leadwire system according to claim 11, wherein said leadwires are shielded.

13. A universal DIN leadwire system according to claim 11, wherein said leadwires are non-shielded.
